(19) United States
(12) Patent Application Publication
(10) Pub. No.: US 2010/0021558 A1
(43) Pub. Date: Jan. 28, 2010

(54) Dilute Aqueous Peracid Solutions and Stabilization Method

(75) Inventors: Emmanuel A. Dada, Bensalem, PA (US); Donald S. Lapham, III, Lockport, NY (US)

Correspondence Address: PATENT ADMINISTRATOR;FMC CORPORATION, 1735 MARKET STREET, PHILADELPHIA, PA, 19103, US

(73) Assignee: FMC CORPORATION, Philadelphia, PA (US)

(21) Appl. No.: 12/507,484

(22) Filed: Jul. 22, 2009

Related U.S. Application Data
(60) Provisional application No. 61/135,850, filed on Jul. 24, 2008.

(51) Int. Cl. A61K 33/40; D06L 3/02

(52) U.S. Cl. 424/616; 8/111

(57) ABSTRACT

A method for stabilizing a dilute peracetic acid solution containing less than about 5 wt % peracetic acid by controlling the concentration of hydrogen peroxide and the mole ratio of hydrogen peroxide to acetic acid and by introducing a stabilizer to provide sequestering activity. The method is particularly useful for the long-term stabilization of dilute peracetic acid solutions. Stabilized dilute peracetic acid solutions are also within the scope of this invention.

### FIELD OF THE INVENTION

The present invention relates to a method for the stabilization of dilute peracid solutions and, more particularly and preferably, to a method for stabilizing dilute peracetic acid solutions and such stabilized peracetic acid solutions.

### BACKGROUND OF THE INVENTION

Peracetic acid, sometimes called peroxyacetic acid or PAA, is a well known chemical for its strong oxidizing potential. Peracetic acid has a molecular formula of C₂H₄O₃ or CH₃COOOH, a molecular mass of 76.05 g/mol, and a molecular structure as follows:

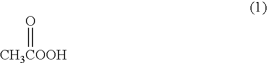

Peracetic acid is a liquid with an acrid odor and is normally sold in commercial formulations as aqueous solutions typically containing, e.g., 5, 15 or 35 wt % peracetic acid. Such aqueous formulations not only contain peracetic acid but also hydrogen peroxide (e.g. 7-25 wt %) and acetic acid (e.g., 6-39 wt %) in a dynamic chemical equilibrium.

Aqueous solutions of peracetic acid, diluted to concentrations below 5 wt % peracetic acid, are widely used in a variety of end use applications for their wide spectrum antimicrobial and biocidal properties, as bactericides, fungicides, disinfectants and sterilants, and also for their bleaching properties. Aqueous peracetic acid exhibits antimicrobial activity that is more potent than aqueous hydrogen peroxide at equivalent low concentrations. A good overview of peracetic acid and its commercial antimicrobial applications is given by M. Kitis in “Disinfection of wastewater with peracetic acid: a review” Environment International 30 (2004) 47-55.

Aqueous peracid (also called peroxyacid) solutions, including aqueous peracetic acid, are susceptible to decomposition, particularly at elevated temperatures, at alkaline pH values and in the presence of impurities, e.g. transition metal ions. The stability of aqueous peracetic acid solutions and other peracid solutions is typically improved by the addition of known hydrogen peroxide or peracid stabilizers. Stabilizers used for stabilization of peracid solutions include pyrophosphoric acid or a pyrophosphate (U.S. Pat. No. 2,347,434 of Reichert et al.), phosphates (U.S. Pat. No. 2,590,856 of Greenspan et al.), phosphonates (GB 925 373 of Henkel GmbH), dipicolinic acid (U.S. Pat. No. 2,609,391 of Greenspan et al.), and tin compounds that are preferably stannates (EP-B1-0 563 584 of Degussa AG).

Other peracid stabilization systems, based on these and other stabilizers, have been described in the literature.

Greenspan et al., in Proc. 42nd Ann. Mtg. Chem. Spec. Man. Assn. December 1955, pp. 59-64, concerns peracetic acid aerosols useful in bacteriological applications and discloses that peracetic acid is considerably less stable than hydrogen peroxide. The reference teaches that dilute peracetic acid solutions present special stability problems and that dilute, e.g. 1%, peracetic acid prepared by dilution of concentrated peracetic acid with water is not stable beyond a few days. Greenspan et al. disclose that stable dilute peracetic acid solutions can be made by use of peracid stabilizers in conjunction with proper adjustment of the relative concentrations of the components of the dilute peracid solution but provide no examples. A typical peracetic acid formulation used in the aerosol work was said to contain 1.0% peracetic acid, 14.5% acetic acid, 5.0% hydrogen peroxide, 1.0% sulfuric acid and 78.5% water.

U.S. Pat. No. 4,015,058 of Böwing et al. discloses stable peroxy-containing concentrates useful for the production of microbicidal agents consisting essentially of an aqueous mixture of 0.5-20 wt % peracetic acid and/or acetic acid or, alternatively, perpropionic acid and/or propionic acid, 25-40 wt % hydrogen peroxide, 0.25-10 wt % organic phosphonic acid capable of sequestering bivalent metal cations and their water-soluble acid salts and, optionally, up to 5 wt % anionic surface-active compounds of the sulfonate and sulfate type. U.S. Pat. No. 4,015,059 of Böwing et al. discloses similar peroxy-containing concentrate compositions similar to those of Böwing et al. '058 except that the sequestering agent component of the '058 patent is omitted.

U.S. Pat. No. 5,656,302 of Cosentino et al. discloses a stable shippable microbicidal composition including between about 0.2 to 8 wt % hydrogen peroxide, about 0.2 to 11 wt % peracetic plus acetic acid, 0 to about 1 wt % sequestrant such as organic phosphonic acid or its salt and water, and 0 to about 1 wt % surfactant, with the ratio of total acid to H₂O₂ being between about 1.0 and 11. The preferred microbicidal formulations contain a considerably greater quantity of peracetic acid plus acetic acid than the quantity of hydrogen peroxide; the hydrogen peroxide concentration is preferably less than about 2 wt % H₂O₂.

Prior art aqueous peracid solutions, even stabilized peracid solution such as those just mentioned, are still susceptible to decomposition losses in long term storage over weeks or months, since ambient temperatures can vary widely and since the presence of even very small amounts of impurities can have an adverse impact during long term storage.

There remains a need for highly stable dilute aqueous peracid solutions that maintain their peracid concentration from the time they are prepared until their ultimate use in various end use applications. An objective of the present invention is the stabilization of dilute aqueous peracid solutions, particularly aqueous peracetic acid, in a manner that provides excellent long term stabilization of such solutions.

### BRIEF SUMMARY OF THE INVENTION

One embodiment of the present invention is a method of stabilizing a dilute peracid solution comprising adjusting the concentration of hydrogen peroxide in an aqueous organic peracid solution, containing a peroxycarboxylic acid, hydrogen peroxide, and corresponding carboxylic acid and, further, containing at least about 0.05 to less than about 5 wt % of the organic peroxycarboxylic acid, to provide a hydrogen peroxide concentration of less than about 20 wt % H₂O₂ and a mole ratio of hydrogen peroxide to carboxylic acid in excess of at least about 1.5:1 hydrogen peroxide:carboxylic acid; and introducing a stabilizer into the aqueous peracid solution, in an amount of about 0.05 wt % to about 5 wt % stabilizer based on the weight of the peracid solution.

Another embodiment of the present invention is a method of stabilizing a dilute peracetic acid solution comprising adjusting the concentration of hydrogen peroxide in an aqueous peracetic acid solution, containing hydrogen peroxide, acetic acid and at least about 0.05 to less than about 5 wt % peracetic acid, to provide a hydrogen peroxide concentration of less than about 20 wt % H₂O₂ and a mole ratio of hydrogen peroxide to acetic acid of at least about 1.5:1 H₂O₂:CH₃COOH; and introducing a stabilizer into the aqueous peracetic acid solution, in an amount of about 0.05 wt % to about 5 wt % stabilizer based on the weight of the peracid solution.

A preferred embodiment of the present invention is a method of stabilizing a dilute peracetic acid solution comprising adjusting the concentration of hydrogen peroxide in an aqueous peracetic acid solution, containing hydrogen peroxide, acetic acid and about 0.2 to about 3 wt % peracetic acid, to provide a hydrogen peroxide concentration of about 2 wt % to less than about 10 wt % H₂O₂ and a mole ratio of hydrogen peroxide to acetic acid of at least about 1.5:1 H₂O₂:CH₃COOH; and introducing a stabilizer comprising 1-hydroxy ethylidene-1,1-diphosphonic acid into the aqueous peracetic acid solution, the stabilizer being introduced in an amount of about 0.1 to about 3 wt % based on the weight of the solution.

Still another preferred embodiment is a stabilized dilute aqueous peracetic acid solution comprising peracetic acid, hydrogen peroxide and acetic acid and containing about 0.5 to about 3 wt % peracetic acid, about 2 wt % to less than about 10 wt % hydrogen peroxide, and a stabilizer in an amount of about 0.1 to about 3 wt % based on the weight of the solution, wherein the mole ratio of hydrogen peroxide to acetic acid is at least about 1.5:1 H₂O₂:CH₃COOH.

### DETAILED DESCRIPTION OF THE INVENTION

The method of the present invention provides a highly effective means for stabilizing dilute aqueous peracetic acid solutions, and other dilute aqueous peracid solutions, from decomposition over extended period of storage. Peracetic acid and other peracid solutions stabilized according to the method of this invention exhibit excellent stability, even over months-long storage periods and/or when subjected to storage at elevated temperatures.

Peracetic acid solutions, the preferred peracid solution in the stabilization method of this invention, are susceptible to loss of their active oxygen by any of several routes, as shown in the following reactions:

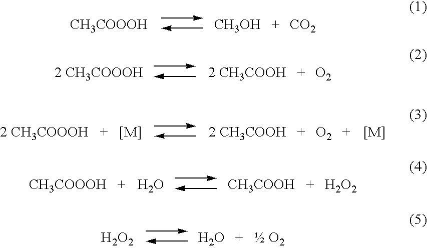

Reactions (1) and (2) represent a spontaneous decomposition (i.e., loss) of peracetic acid. Reaction (3) is the metal [M]-catalyzed, i.e., impurity-catalyzed, decomposition of peracetic acid. Reaction (4) is the hydrolysis decomposition of peracetic acid.

Reaction (5) is the decomposition reaction for hydrogen peroxide that normally present in an aqueous peracetic acid or other peracid solution. Loss of hydrogen peroxide in an aqueous peracid solution can upset the equilibrium concentrations of peracid and hydrogen peroxide in such a solution, leading to a consequent loss of peracid as the equilibrium is reestablished.

The multiple routes for loss of active oxygen, indicated above, confirm that (prior art) stabilization techniques directed to just one of the specific reactions noted above may not be effective at ensuring excellent stabilization of the aqueous peracid solution from decomposition losses. It should be noted that many prior art approaches described in the literature, for stabilization of peracid solutions against loss of activity, have typically focused on shorter term stability, e.g. for periods of a few days or weeks, rather than extended periods of up to a year or more. Likewise, prior art stabilization techniques are typically not evaluated in reported examples against stringent temperature conditions, above 40° C. or, more preferably, at 50° C. or higher, for demonstration of their utility in real-world storage conditions.

The present invention is directed to the stabilization of dilute aqueous solutions of a peracid, which is preferably peracetic acid, by adjustment of the absolute and relative amounts of the components in the aqueous peracid solutions, and by the incorporation of a stabilizer. The stabilization method is characterized by providing excellent peracid solution stability for dilute stabilized peracid solutions, even over extended storage times and under extreme ambient temperature storage conditions.

The simplicity and straightforwardness of the stabilization method of this invention, with its ability to quickly provide highly stable dilute peracid solutions, underscores the significant advance in the art afforded by this method.

### Suitable Organic Peracids

Peracetic acid (peroxyacetic acid) is the most preferred peracid for stabilization in the method of the present invention, but the stabilization method is likewise applicable to numerous other organic peracids that are water-soluble or water-miscible.

Other organic peracids (also called peroxyacids) suitable for use in the method of this invention include one or more C₁ to C₁₂ peroxycarboxylic acids selected from the group consisting of monocarboxylic peracids and dicarboxylic peracids, used either individually or in combinations of two, three or more peracids. The peroxycarboxylic acid is preferably a C₂ to C₅ peroxycarboxylic acid selected from the group consisting of monocarboxylic peracids and dicarboxylic peracids. The peracid should be at least partially water-soluble or water-miscible.

One preferred category of suitable organic peracids includes peracids of a lower organic aliphatic monocarboxylic acid having 2-5 carbon atoms, such as acetic acid (ethanoic acid), propionic acid (propanoic acid), butyric acid (butanoic acid), iso-butyric acid (2-methyl-propanoic acid), valeric acid (pentanoic acid), 2-methyl-butanoic acid, iso-valeric acid (3-methyl-butanoic) and 2,2-dimethyl-propanoic acid. Organic aliphatic peracids having 2 or 3 carbon atoms, e.g. peracetic acid and peroxypropanoic acid, are preferred.

Another category of suitable lower organic peracids includes peracids of a dicarboxylic acid having 2-5 carbon atoms, such as oxalic acid (ethanedioic acid), malonic acid (propanedioic acid), succinic acid (butanedioic acid), maleic acid (cis-butenedioic acid) and glutaric acid (pentanedioic acid).

Peracids having between 6-12 carbon atoms that may be used in the method of this invention include peracids of monocarboxylic aliphatic acids such as caproic acid (hexanoic acid), enanthic acid (heptanoic acid), caprylic acid (octanoic acid), pelargonic acid (nonanoic acid), capric acid (decanoic acid) and lauric acid (dodecanoic acid), as well as peracids of monocarboxylic and dicarboxylic aromatic acids such as benzoic acid, salicylic acid and phthalic acid (benzene-1,2-dicarboxylic acid).

### Concentration of Peracid in Stabilized Peracid Solutions

The aqueous peracid solution stabilized according to the method of this invention is a dilute aqueous peracid solution, preferably containing less than about 5 wt % peracid. Dilute peracetic acid solution, the preferred stabilized peracid solution, stabilized by the method of this invention preferably contains less than about 5 wt % peracetic acid.

Preferred embodiments of this invention are stabilized peracetic acid solutions containing up to about 3 wt % peracetic acid.

The minimum concentration of peracetic acid or other peracid in the dilute stabilized peracid solution of this invention is preferably at least about 0.05 wt % peracid.

More preferably, the peracid concentration in the dilute stabilized solution is at least about 0.1 wt % peracid. In the case of peracetic acid solutions, the peracetic acid concentration is even more preferably at least about 0.2 wt % peracetic acid and most preferably at least about 0.5 wt % peracetic acid.

The method of this invention is particularly preferred for stabilization of dilute peracetic acid solutions containing about 0.5 wt % to about 3 wt % peracetic acid. This latter concentration range is especially useful for highly stabilized peracetic acid solutions used in consumer, medical and food service applications.

In addition, the stabilized dilute peracetic acid solution is preferably in substantial equilibrium with respect to its solution components, and this aspect is discussed in more detail below.

### Concentration of Hydrogen Peroxide in Stabilized Peracid Solutions

The stabilization method of this invention requires that the concentration of hydrogen peroxide in the stabilized peracid solution be adjusted to be less than about 20 wt % H₂O₂.The minimum concentration of hydrogen peroxide in the stabilization method of this invention should be sufficient to provide a significant, substantial level of this component in the stabilized peracid solution. The hydrogen peroxide concentration in the stabilized peracid solution is preferably at least about 2 wt % H₂O₂.

Such significant, substantial concentrations of hydrogen peroxide are one aspect of the present invention that provides enhanced long-term stability of the stabilized peracid solutions of this invention. By way of example, if a 0.5 wt % peracetic acid solution with 9.1 wt % H₂O₂ were to experience a loss of 1 wt % H₂O₂, the reequilibrated peracetic solution would lose about 10% of its initial peracetic acid concentration, resulting in the peracetic acid solution containing 0.45 wt % peracetic acid and 8.1 wt % H₂O₂. By contrast, if a 0.5 wt % peracetic acid solution with only 2.5 wt % H₂O₂ were to experience a loss of 1 wt % H₂O₂, the reequilibrated peracetic solution would lose about 40% of its initial peracetic acid concentration, resulting in the peracetic acid solution containing 0.3 wt % peracetic acid and 0.5 wt % H₂O₂.

The concentration of hydrogen peroxide in the aqueous peracid solution is preferably adjusted to provide less than about 15 wt % H₂O₂. More preferably, the concentration of hydrogen peroxide in the aqueous peracid solution, particularly in peracetic acid solutions, is adjusted to provide less than about 10 wt % H₂O₂ and, most preferably, less than about 8 wt % H₂O₂. One advantage of having the concentration of hydrogen peroxide maintained at less than about 8 wt % H₂O₂ is that such solutions are not regulated by the U.S. Department of Transportation as stringently as solutions containing 8-20 wt % H₂O₂.

The hydrogen peroxide used as a component in the stabilization method of this invention, to adjust the concentration of hydrogen peroxide in the stabilized peracid solution, is normally concentrated hydrogen peroxide. Highly purified grades of hydrogen peroxide are preferred as the source of hydrogen peroxide used to formulate the dilute peracid solutions in the method of this invention.

In the stabilization method of this invention, the hydrogen peroxide source (used to adjust the H₂O₂ concentration in the stabilized peracid solution) will typically be used at a concentration in the range from about 20 wt % H₂O₂ to about 70 wt % H₂O₂, but more dilute concentrations of hydrogen peroxide may also be used, e.g. about 5 up to about 20 wt % H₂O₂. In any event, the water included in such aqueous hydrogen peroxide solution must be taken into account, so that the concentrations of all components in the dilute stabilized peracid solution are at the desired concentrations after addition of the aqueous hydrogen peroxide.

### Ratio of Hydrogen Peroxide to Acetic Acid (or Other Carboxylic Acid)

The method of stabilizing peracid solutions in this invention also involves adjustment of the mole ratio of hydrogen peroxide to carboxylic acid to a value of at least about 1.5:1 H₂O₂:carboxylic acid, to maintain a significant, substantial hydrogen peroxide concentration in the aqueous peracid solution. References to carboxylic acid in this specification are intended to mean, in the case of a specific percarboxylic acid peracid, the counterpart carboxylic acid for such peracid; e.g. in the case of peracetic acid, acetic acid, in the case of perpropionic acid, propionic acid, and the like.

For stabilization of aqueous peracetic acid, the preferred peracid, the concentration of hydrogen peroxide in the peracetic acid solution is preferably adjusted to provide a mole ratio of hydrogen peroxide to acetic acid of at least about 2:1 H₂O₂:acetic acid.

As noted above, the stabilization method of the present invention requires the presence of a significant concentration of hydrogen peroxide (but less than 20 wt % H₂O₂), as compared to the concentration of carboxylic acid, and this is achieved by adjusting the mole ratio of hydrogen peroxide to carboxylic acid in the stabilized peracid solution to be at least about 1.5:1. The inventors have discovered that this mole ratio requirement, another factor ensuring the presence of a relatively substantial hydrogen peroxide concentration, serves to minimize the rate of decomposition of peracid, as compared to analogous solutions containing relatively low molar ratios of hydrogen peroxide: carboxylic acid, i.e., less than 1.5:1.

### Acetic Acid (or Other Carboxylic Acid)

The carboxylic acid component of the peracid solution stabilized by the method of this invention is preferably utilized as a relative pure grade of carboxylic acid, where adjustment of its concentration requires addition of the acid. In the case of peracetic acid, the preferred peracid, adjustment of the acetic acid concentration in the stabilized peracetic acid solution may be carried out, as necessary, with purified glacial acetic acid, a water-free acetic acid. Acetic acid, also known as ethanoic acid and having the chemical formula CH₃COOH, is a widely available chemical reagent and is considered a weak acid. Acetic acid is corrosive and an irritant, so appropriate safety and handling measures must be employed in its transport, storage and handling.

### Stabilizing Agent

A stabilizer is introduced into the aqueous peracid solution in the method of this invention to maintain the equilibrated peracid concentration over extended periods of time. The stabilizer is typically a compound or compounds or agent whose sequestering (e.g., chelating or complexing) activity stabilizes the aqueous peracid solution and its active oxygen content against decomposition from impurities present in the aqueous solution. Without the presence of the stabilizer, such impurities may otherwise have an adverse or deleterious effect on the stability of the peracid and/or hydrogen peroxide in the solution, even at extremely low concentrations of such impurities.

The impurities typically include materials that react with the peracid and/or hydrogen peroxide in the aqueous peracid solution or that catalyze their decomposition. Such impurities may originate from the water source used to prepare the aqueous peracid solution or from materials of construction of containers, reaction vessels or process piping used in the preparation and/or storage of the peracid solution.

Examples of such impurities include metal ions, metals and metal compounds (e.g., oxides, hydroxides or sulfides), particularly those of the transition metals, including the heavy metals. These impurities may be present in the aqueous solution either in dissolved or suspended form.

Metal impurities that can have an adverse effect on the stability of aqueous peracid solutions include iron, nickel, copper, zinc, manganese, mercury, chromium, cobalt, cadmium, silver, platinum and the like. Some combinations of these metals exhibit more than their individual catalytic effects alone, e.g., iron and copper.

The stabilizer is introduced into the aqueous peracid solution in an amount that is sufficient to provide sequestering activity in the aqueous peracid solution. The stabilizer is preferably introduced into the peracid solution in an amount of about 0.05 wt % to about 5 wt % stabilizer, based on the weight of the aqueous peracid solution. More preferably, the amount of stabilizer introduced into the peracid solution is about 0.1 wt % to about 3 wt % stabilizer, based on the weight of the aqueous peracid solution.

The amount of stabilizer introduced into the preferred peracid solution, aqueous peracetic acid, is preferably about 0.1 wt % to about 3 wt % stabilizer, based on the weight of the aqueous peracetic acid solution.

The upper limit for the amount of stabilizer introduced is normally limited only by practical and economic constraints, since the stabilizer is typically one of the most costly components of the overall peracid composition. The amount of stabilizer used in peracid solutions, including peracetic acid solutions, is therefore desirably minimized, while still providing the desired long term stability that is a characteristic of this invention.

For this reason, the stabilizer concentration of the stabilized peracetic acid solutions of this invention is most preferably maintained within the range of about 0.1 wt % to about 1 wt % stabilizer, based on the weight of the peracetic acid solution.

The optimum amount of stabilizing agent required, used to sequester or complex or chelate impurities present in the aqueous peracid solution, can often be minimized by ensuring that relatively pure components and/or purified water are utilized in the preparation of the peracid solution. The water source utilized for preparation of the peracetic acid or other aqueous peracid solution in the method of this invention is therefore preferably relatively pure, and the purified water source is preferably selected from the group consisting of deionized water and distilled water. Likewise, purified components are preferably used for the preparation of the peracetic acid solution.

Stabilizing agents suitable for use in the method of this invention include stabilizers conventionally used for stabilization of aqueous hydrogen peroxide solutions. Suitable stabilizing agents include the following water-soluble compounds:
(i) organic phosphonic acids and their salts, such as organopolyphosphonic acids and their salts, including hydroxyethylidenediphosphonic acids and their salts. A preferred hydroxyethylidenediphosphonic acid is 1-hydroxy ethylidene-1,1-diphosphonic acid (commonly called HEDP);
(ii) amine-substituted phosphonic acids, such as dimethyl amino methane diphosphonic acid; and alkyleneaminomethylene phosphonic acids such as ethylene diaminotetra methylene phosphonic acid (EDTMPA), cyclohexane-1,2-diaminotetramethylene phosphonic acid and diethylenetriaminepenta methylene phosphonic acid (DTPMPA);
(iii) carboxylic acid substituted N-containing heterocyclics, including dipicolinic acid (DPA) and picolinic acid and their salts, and hydroxyquinoline, i.e., 8-hydroxyquinoline;
(iv) aminopolycarboxylic acids and polyaminocarboxylic acids and their salts, including ethylenediaminetetraacetic acid (EDTA) and diethylenetriaminepentaacetic acid (DTPA); triethylenetetraminehexaacetic acid (TTHA);
(v) tin-based compounds such as potassium stannate and sodium stannate;
(vi) phosphoric acids and phosphates such as organic phosphoric acids and their salts, and sodium pyrophosphate;
(vii) alkylbenzene sulfonates (with 6-18 carbon atoms); and alkyl sulfates.

Highly preferred for use in the stabilization of the preferred peracid, peracetic acid, is a stabilizing agent comprising 1-hydroxy ethylidene-1,1-diphosphonic acid (HEDP). A commercially-available stabilizer comprising 1-hydroxy ethylidene-1,1-diphosphonic acid is Dequest® 2010 (Thermphos International B.V., Vlissingen-Oost, NL). Other preferred stabilizers include dipicolinic acid, and sodium and potassium stannates.

Suitable stabilizing agents include combinations of two or more stabilizing agents, e.g. an organophosphonate and an N-heterocyclic carboxylic acid being one particularly suitable combination and sodium stannate and sodium pyrophosphate being another.

### Equilibrium Peracid Solutions

The method of this invention is preferably carried out such that the stabilized aqueous peracid is equilibrated, i.e., in substantial equilibrium with respect to its solution components, upon completion of the method steps. This may be accomplished by suitable selection and adjustment of the respective peracid solution components, as described below.

The aqueous peracid solution, after completion of the stabilization procedure of this invention, preferably contains a peracid (peroxycarboxylic acid) that is equilibrated with respect to the hydrogen peroxide, corresponding carboxylic acid and water components that are also present in the aqueous solution. In the case of a stabilized dilute peracetic acid (the preferred peracid) solution, the aqueous solution components that are equilibrated with each other are peracetic acid, hydrogen peroxide, acetic acid and water.

The terms “equilibrated’ and “in substantial equilibrium” are intended to refer to peracid solutions in which the peracid concentration is within ±10% of the equilibrium concentration and, more preferably, within ±5% of the equilibrium peracid concentration.

Determination of the approximate equilibrium composition of the specific stabilized peracetic acid solution or other peracid solution that is desired may be obtained by prior knowledge, e.g. published peracetic acid compositions.

Alternatively, the equilibrium composition of the desired stabilized peracetic acid solution or other peracid solution may be determined empirically, e.g., by water or aqueous hydrogen peroxide dilution of a more concentrated peracid solution to the approximate peracid concentration sought and then allowing the solution to reach equilibrium, before analysis of the individual component concentrations is carried out.

After the experimental aqueous peracid solution has reached equilibrium with respect to its peracid, hydrogen peroxide, corresponding carboxylic acid and water components, the concentration levels of each component may be analyzed, to determine the precise composition of the equilibrated solution. Although this empirical technique requires some length of time for an equilibrated solution to be achieved, this procedure need only be carried out once to obtain the equilibrium composition parameters.

The resulting information, i.e., the concentrations of the individual components in the desired equilibrated peracetic acid solution, may be used in the future to calculate the amounts of each component required to prepare the desired stabilized aqueous peracid solution having a specific peracid concentration.

### Stabilized Peracid Solution: pH and Temperature

The pH of the peracetic acid solution or other peracid solution is not critical in the method of this invention, but the pH of the peracetic acid solution or other peracid solution should be acidic or neutral. Decomposition of a peracid such as peracetic acid is more likely to occur in basic solutions, particularly at a pH value more basic than pH 8, so peracetic acid solutions with acidic pH values are preferred to promote enhanced long-term stability of the peracetic acid.

For peracetic acid solutions stabilized according to the method of this invention, no pH adjustment is normally required since the peracetic acid solution pH is typically acidic. Stabilized peracetic acid solutions containing about 1 wt % peracetic acid will typically exhibit a pH of about 1-3.

The temperature at which the peracid stabilization method is carried out in the method of this invention is not critical. Temperatures during the stabilization method of about 5° C. to about 80° C. are feasible, with temperatures in the range of about 10° C. to about 50° C. being preferred.

The temperature at which the stabilized peracid solution is maintained, after stabilization according to the method of this invention, is preferably within the range of about 10° C. to about 30° C. It should be understood that deviations from this preferred range may occur in commercial storage facilities or during transport, but that such deviations will normally not adversely impact the long-term stability of the peracid solution against peracid decomposition. The Examples presented below illustrate the excellent stability of peracetic acid stabilized according to the method of this invention, even when such peracid solutions are subjected to a stringent constant storage temperature of 50° C. for months.

The method of the present invention, for the stabilization of peracetic acid or other peracid solutions, may be carried out on a continuous basis, including semi-continuous, or as a batch wise operation. In any of the continuous, semi-continuous or batch wise operations, the method of this invention may be implemented without the need for specialized equipment and may be carried out at ambient temperatures and pressures.

Batch wise operation is favored where preparation of a small-to-moderate quantity of stabilized peracid is desired. Continuous operation of the method of this invention is particularly useful for preparation of large quantities of stabilized peracid solution.

The components used to prepare the dilute aqueous peracid solution are introduced into aqueous solution with agitation or mixing sufficient to provide rapid dispersion of the diluent components and produce a homogeneous mixture of the components throughout the peracid solution. Such mixing/agitation may be provided via conventional means, e.g. stirred tank, inline fluid mixing, or the like. The components used to prepare the dilute aqueous peracid solution may be introduced concurrently, sequentially or as previously combined components.

### Long-Term Stability of Peracid Solution

The stability of the aqueous peracid solutions of this invention is noteworthy for its long duration. Long term storage stability refers to the aqueous peracid solutions of this invention retaining their initial equilibrated peracid concentration over extended periods of time, e.g. over many months. The method of the present invention provides dilute peracetic acid solutions that exhibit unusually good storage stability, retaining at least about 80%, preferably at least about 90%, of the initial equilibrated peracetic acid concentration for at least six months under typical commercial storage conditions. Depending on storage conditions, such peracetic acid solutions may exhibit good storage stability for at least nine months and even twelve months or longer.

Stabilized peracetic acid produced by the method of this invention has wide applicability as a disinfecting, sterilizing, biocidal or antimicrobial agent in both commercial and consumer applications. Commercial or industrial applications include the food processing, beverage, pharmaceutical and medical industries, industrial waste water, and use as a bleaching agent in the textile, pulp and paper industries. Consumer applications include laundry and bleaching uses.

### Diluted Peracid Solutions

Peracetic acid end uses involving disinfecting, sanitizing, biocidal or antimicrobial applications may call for very dilute peracetic acid equilibrium concentrations, typically less than about 1 wt % peracetic acid and, more typically, less than about 0.1 wt % (1000 ppm) peracetic acid. Very dilute concentrations of peracetic acid may be prepared directly in the stabilization method of this invention. Alternatively, such dilute solutions may be prepared by dilution as needed (e.g., with water) of a more concentrated stabilized peracetic acid solution obtained in the method of this invention.

The concentration of the peracetic acid in some end-use applications (e.g., when diluted by its addition to an aqueous medium being treated) can be as low as about 1-10 ppm and still provide the desired activity, e.g. disinfecting, sanitizing, biocidal, antimicrobial (including industrial waste water treatment) or bleaching activity. Studies have shown that peracetic acid is very active even at very low concentrations, e.g. as low as 1 or 2 ppm. Low peracetic acid concentrations of about 1-10 ppm, for example, can provide disinfecting activity that accomplishes the desired disinfecting objective within minutes.

These highly dilute peracetic acid solutions may be prepared on-site, for immediate use, via water dilution of a stabilized (and preferably equilibrated) peracetic acid solution obtained by the method of this invention. When diluted and immediately used on-site, the water-diluted aqueous peracetic acid solution is not equilibrated, but its immediate utilization in an end-use application makes the lack of equilibrium immaterial. This approach is useful for the on-site preparation of very dilute peracetic acid solutions, e.g., containing less than about 0.01 wt % (100 ppm) peracetic acid.

### EXAMPLES

The following non-limiting Examples illustrate preferred embodiments of the present invention.

### Example 1

Example 1 is a laboratory-scale study in which three dilute aqueous peracetic acid solutions were prepared according to the method of this invention, with low stabilizer levels ranging from 0.05 wt % to 0.2 wt % stabilizer. Two samples of each of the three peracetic acid solutions were respectively evaluated for their long term stability while being maintained at constant storage temperatures of 25° C. and 50° C.

The aqueous peracetic acid solutions contained about 0.5-0.6 wt % peracetic acid, about 9 wt % hydrogen peroxide and about 5 wt % acetic acid, with three different concentration levels of a stabilizer. The stabilizer was a commercially-available stabilizer comprising 1-hydroxy ethylidene-1,1-diphosphonic acid, namely Dequest® 2010 stabilizer, which is marketed by Thermphos International B.V., Vlissingen-Oost, NL. The amounts of Dequest 2010 stabilizer in the aqueous peracetic acid solutions in the three studies were 0.05 wt %, 0.1 wt % and 0.2 wt % stabilizer, all percentages based on the total weight of the aqueous peracetic acid solution.

The dilute aqueous peracetic acid solutions of this Example were prepared in laboratory-scale equipment by dilution of a concentrated peracetic acid formulation containing about 16 wt % peracetic acid and 10 wt % hydrogen peroxide. The concentrated peracetic acid solution was made by combining appropriate amounts of peracetic acid, acetic acid, 72.2 wt % hydrogen peroxide, Dequest® 2010 stabilizer and deionized water.

An analysis of the concentrated aqueous peracetic acid solution prepared as described above indicated the following composition (all values in wt %):

| | |
|---|---|
| peracetic acid | 16.2 |
| hydrogen peroxide | 9.6 |
| acetic acid | 34.8 |
| stabilizer | 0.62 |
| water (free) | 38.8 |

This concentrated 16 wt % peracetic acid solution was then used to prepare the dilute peracetic acid solutions of this Example, by dilution of the concentrated peracetic acid solution with additional acetic acid, 72.2 wt % hydrogen peroxide and water and by addition of an appropriate amount of Dequest® 2010 stabilizer.

The concentrated peracetic acid solution, in an amount of 15 wt units, was combined with 17.7 wt units of acetic acid, 54.0 wt units of hydrogen peroxide (72.2 wt %), 358.3 wt units of deionized water, and 0.20 wt units of Dequest® 2010 stabilizer to prepare a dilute peracetic acid solution containing 0.5-0.6 wt % peracetic acid with 0.05 wt % stabilizer in Example 1A.

In Examples 1B and 1C, the dilute peracetic acid solutions contained 0.5-0.6 wt % peracetic acid with 0.1 and 0.2 wt % stabilizer, respectively, so the amounts of diluent water and stabilizer were modified to be 357.9 wt units of water and 0.56 wt units of stabilizer (Example 1B) and 357.2 wt units of water and 1.26 wt units of stabilizer (Example 1C), respectively.

The stabilized dilute peracetic acid solutions were prepared by adding the requisite stabilizer to the water, with mixing, and then likewise adding the requisite acetic acid (as glacial acetic acid), hydrogen peroxide (as 72.2 wt % H₂O₂), and then the concentrated 16 wt % peracetic acid solution. This preparation procedure was carried out at a temperature of 25° C.

This general procedure was also used in subsequent Examples for the preparation of stabilized dilute peracetic acid solutions described in those Examples.

Each of the dilute peracetic acid solutions in Examples 1A, 1B and 1C, as mentioned above, contained about 0.5-0.6 wt % peracetic acid, about 9 wt % hydrogen peroxide and about 5 wt % acetic acid. The mole ratio of hydrogen peroxide to acetic acid of these peracetic acid solutions was therefore about 3.1:1. The compositions of the dilute peracetic acid solutions of Examples 1A, 1B and 1C were analyzed after their preparation, for their peracetic acid, hydrogen peroxide and acetic acid concentrations, and these analyses are shown in Tables 1A, 1B and 1C, in the first data row of each Table.

Separate samples of the dilute peracetic acid solutions were evaluated at two constant storage temperatures, 25° C. and 50° C., for an extended period lasting over four months. The dilute peracetic acid solution samples were periodically analyzed to determine their concentrations of peracetic acid, hydrogen peroxide and acetic acid during the storage period. The dilute peracetic acid solution samples maintained at 25° C. showed excellent stability, with the composition analyses exhibiting essentially unchanged levels of peracetic acid, hydrogen peroxide and acetic acid over a period of 132 days.

It should be noted that the more stringent constant temperature test at 50° C. was intended to duplicate a high temperature that may be experienced intermittently for commercial peracetic acid solutions in actual real-world storage conditions. In real-world storage conditions, storage temperatures are likely to fluctuate, with a 50° C. temperature being a realistic high (maximum) temperature. However, such very high temperatures are not likely to be maintained as a constant temperature, particularly over the extended time periods studied in these Examples. But such constant temperature long term storage testing, at a stringent temperature of 50° C., is useful for evaluating the relative stability of the various stabilized peracetic acid solutions prepared in these Examples.

The periodic analyses of the dilute peracetic acid solutions maintained at a storage temperature of 50° C. are shown for Examples 1A, 1B and 1C in Tables 1A, 1B and 1C, respectively, for the 132 day storage stability test period.

TABLE 1A
Long Term Stability Evaluation at 50° C.:
Aqueous 0.52 wt % Peracetic Acid Solution
Containing 0.05 wt % Dequest ® Stabilizer

| Days | Peracetic Acid | H₂O₂ | Acetic Acid |
|---|---|---|---|
| after Preparation | wt % | wt % | wt % |
| 0 | 0.52 | 9.1 | 5.1 |
| 5 | 0.59 | 9.1 | 5.1 |
| 13 | 0.51 | 9.1 | 5.2 |
| 55 | 0.50 | 9.0 | 5.1 |
| 68 | 0.51 | 8.9 | 5.2 |
| 105 | 0.49 | 8.7 | 5.2 |
| 132 | 0.44 | 8.2 | 5.3 |

TABLE 1B
Long Term Stability Evaluation at 50° C.:
Aqueous 0.54 wt % Peracetic Acid Solution
Containing 0.1 wt % Dequest ® Stabilizer

| Days | Peracetic Acid | H₂O₂ | Acetic Acid |
|---|---|---|---|
| after Preparation | wt % | wt % | wt % |
| 0 | 0.54 | 9.1 | 5.1 |
| 5 | 0.53 | 9.0 | 5.1 |
| 13 | 0.50 | 9.0 | 5.1 |
| 33 | 0.54 | 9.0 | 5.2 |
| 68 | 0.54 | 8.9 | 5.2 |
| 106 | 0.51 | 8.8 | 5.2 |
| 132 | 0.51 | 8.6 | 5.2 |

TABLE 1C
Long Term Stability Evaluation at 50° C.:
Aqueous 0.55 wt % Peracetic Acid Solution
Containing 0.2 wt % Dequest ® Stabilizer

| Days | Peracetic Acid | H₂O₂ | Acetic Acid |
|---|---|---|---|
| after Preparation | wt % | wt % | wt % |
| 0 | 0.55 | 9.1 | 5.1 |
| 5 | 0.55 | 9.4 | 5.1 |
| 13 | 0.54 | 9.0 | 5.1 |
| 33 | 0.55 | 9.0 | 5.2 |
| 68 | 0.55 | 8.9 | 5.3 |
| 106 | 0.55 | 8.7 | 5.2 |
| 132 | 0.52 | 8.5 | 5.3 |

The dilute peracetic acid solution samples maintained at 50° C., the more stringent stability test temperature, demonstrated very good stability at the lowest stabilizer concentration of 0.05 wt % stabilizer and excellent stability for stabilizer concentrations of 0.1 wt % and 0.2 wt %. The peracetic acid solution in Example 1A, which was a 0.52 wt % peracetic acid solution with 0.05 wt % stabilizer, retained about 85% of its initial peracetic acid concentration (and about 90% of the initial hydrogen peroxide concentration) after 132 days of the solution being maintained at a storage temperature of 50° C.

The peracetic acid solutions in Examples 1B and 1C, which were 0.54-0.55 wt % peracetic acid solutions with 0.1 wt % and 0.2 wt % stabilizer, respectively, retained about 94% of the initial peracetic acid concentration (and over 90% of the initial hydrogen peroxide concentrations) after 132 days of the solutions being maintained at a storage temperature of 50° C.

The analyses for these Examples also confirm that the dilute peracetic acid solutions were essentially equilibrium solutions, with respect to their peracetic acid, hydrogen peroxide and acetic acid components in the initial dilute peracetic solutions, since there was no significant shift in the respective proportions and amounts of these components in the solution over the first weeks of the test period.

### Example 2

Example 2 is a another laboratory-scale study in which three dilute aqueous peracetic acid solutions were prepared according to the method of this invention, with higher stabilizer levels ranging from 0.3 wt % to 0.9 wt % stabilizer, and separate samples were evaluated for their long term stability while being maintained at constant temperatures of 25° C. and 50° C.

The aqueous peracetic acid solutions contained about 0.7-0.8 wt % peracetic acid, about 9 wt % hydrogen peroxide and about 5 wt % acetic acid, with three different concentration levels of a stabilizer being used. The stabilizer in this Example 2 was Dequest 2010 stabilizer comprising 1-hydroxy ethylidene-1,1-diphosphonic acid, the same stabilizer used in Example 1. The aqueous peracetic acid solutions in the three studies, Examples 2A, 2B and 2C, respectively contained concentrations of Dequest® 2010 stabilizer of 0.3 wt %, 0.6 wt % and 0.9 wt % stabilizer, all percentages based on the total weight of the aqueous peracetic acid solution.

The aqueous peracetic acid solutions in the three studies, Examples 2A, 2B and 2C, were prepared according to the general procedure described in Example 1. In addition, Examples 2B and 2C also contained sulfuric acid, introduced at a concentration of 1 wt % H₂SO₄. The compositions of the dilute peracetic acid solutions of Examples 2A, 2B and 2C were analyzed after their preparation for their peracetic acid, hydrogen peroxide and acetic acid concentrations, and these analyses are respectively shown in Tables 2A, 2B and 2C, in the first data row of each Table.

The stabilized dilute peracetic acid solution in Example 2A, with 0.3 wt % stabilizer, contained 0.81 wt % peracetic acid, 9.3 wt % hydrogen peroxide, 5.3 wt % acetic acid and therefore had a mole ratio of hydrogen peroxide to acetic acid of about 3.1:1. The stabilized dilute peracetic acid solution in Example 2B, with 0.6 wt % stabilizer, contained 0.73 wt % peracetic acid, 9.1 wt % hydrogen peroxide, 5.2 wt % acetic acid and also had a mole ratio of hydrogen peroxide to acetic acid of about 3.1:1. The third stabilized dilute peracetic acid solution in Example 2C, with 0.9 wt % stabilizer, contained 0.84 wt % peracetic acid, 9.2 wt % hydrogen peroxide, 5.2 wt % acetic acid and 0.9 wt % stabilizer and also had a mole ratio of hydrogen peroxide to acetic acid of about 3.1:1.

Separate samples of the dilute peracetic acid solutions from Examples 2A, 2B and 2C were evaluated at two constant storage temperatures, 25° C. and 50° C., for an extended period lasting over eight months. The dilute peracetic acid solution samples were periodically analyzed to determine their concentrations of peracetic acid, hydrogen peroxide and acetic acid during the storage period.

Each of the dilute peracetic acid solution samples for Examples 2A, 2B and 2C maintained at 25° C. showed excellent stability, since the samples exhibited essentially unchanged levels of peracetic acid, hydrogen peroxide and acetic acid over a period of 267 days.

The dilute peracetic acid solution samples for Examples 2A, 2B and 2C maintained at 50° C., a more stringent stability test temperature, also demonstrated excellent stability at all three levels of stabilizer used, 0.3 wt %, 0.6 wt % and 0.9 wt % stabilizer. The periodic analyses of the dilute peracetic acid solutions maintained at a storage temperature of 50° C. are shown for Examples 2A, 2B and 2C in Tables 2A, 2B and 2C, respectively, for the 267 day storage stability test period.

TABLE 2A
Long Term Stability Evaluation at 50° C.:
Aqueous 0.8 wt % Peracetic Acid Solution
Containing 0.3 wt % Dequest ® Stabilizer

| Days | Peracetic Acid | H₂O₂ | Acetic Acid |
|---|---|---|---|
| after Preparation | wt % | wt % | wt % |
| 0 | 0.81 | 9.3 | 5.3 |
| 1 | 0.78 | 9.2 | 5.3 |
| 3 | 0.78 | 9.1 | 5.3 |
| 7 | 0.76 | 9.1 | 5.3 |
| 14 | 0.76 | 9.2 | 5.3 |
| 28 | 0.78 | 9.1 | 5.3 |
| 57 | 0.78 | 9.1 | 5.4 |
| 87 | 0.78 | 9.0 | 5.4 |
| 112 | 0.80 | 9.0 | 5.4 |
| 136 | 0.81 | 8.8 | 5.5 |
| 171 | 0.81 | 8.8 | 5.6 |
| 199 | 0.82 | 8.7 | 5.6 |
| 233 | 0.80 | 8.3 | 5.6 |
| 267 | 0.72 | 7.3 | 5.7 |

TABLE 2B
Long Term Stability Evaluation at 50° C.:
Aqueous 0.7 wt % Peracetic Acid Solution
Containing 0.6 wt % Dequest ® Stabilizer

| Days | Peracetic Acid | H₂O₂ | Acetic Acid |
|---|---|---|---|
| after Preparation | wt % | wt % | wt % |
| 0 | 0.73 | 9.1 | 5.2 |
| 1 | 0.70 | 9.2 | 5.3 |
| 3 | 0.72 | 9.1 | 5.2 |
| 7 | 0.68 | 9.1 | 5.2 |
| 14 | 0.70 | 9.2 | 5.3 |
| 28 | 0.68 | 9.1 | 5.3 |
| 57 | 0.71 | 9.0 | 5.3 |
| 87 | 0.69 | 9.0 | 5.4 |
| 112 | 0.71 | 8.9 | 5.4 |
| 136 | 0.71 | 8.7 | 5.4 |
| 171 | 0.69 | 8.6 | 5.5 |
| 199 | 0.72 | 8.6 | 6.0 |
| 233 | 0.75 | 8.4 | 5.6 |
| 267 | 0.73 | 8.3 | 5.6 |

TABLE 2C
Long Term Stability Evaluation at 50° C.:
Aqueous 0.8 wt % Peracetic Acid Solution
Containing 0.9 wt % Dequest ® Stabilizer

| Days | Peracetic Acid | H₂O₂ | Acetic Acid |
|---|---|---|---|
| after Preparation | wt % | wt % | wt % |
| 0 | 0.84 | 9.2 | 5.2 |
| 1 | 0.80 | 9.2 | 5.2 |
| 3 | 0.81 | 9.1 | 5.2 |
| 7 | 0.79 | 9.1 | 5.2 |
| 14 | 0.81 | 9.1 | 5.2 |
| 28 | 0.80 | 9.0 | 5.2 |
| 57 | 0.80 | 8.9 | 5.3 |
| 87 | 0.83 | 8.7 | 5.3 |
| 112 | 0.87 | 8.5 | 5.3 |
| 136 | 0.86 | 8.3 | 5.3 |
| 171 | 0.92 | 8.2 | 5.4 |
| 199 | 0.92 | 8.1 | 5.5 |
| 233 | 0.97 | 7.9 | 5.5 |
| 267 | 1.00 | 7.6 | 5.5 |

In Example 2A, the dilute peracetic acid solution sample containing 0.3 wt % stabilizer showed only a slight decline in peracetic acid concentration at the final analysis 267 days after initial preparation while stored at 50° C., still retaining 89% of the initial peracetic acid concentration (and 78% of the initial hydrogen peroxide concentration).

In Examples 2B and 2C, the dilute peracetic acid solution samples containing 0.6 and 0.9 wt % stabilizer respectively exhibited no drop in their peracetic acid concentrations during their 267 days of storage at 50° C. maintained at 50° C. The peracetic acid concentration in Example 2C actually increased slightly over the test period, as the hydrogen peroxide concentration declined slightly and resulted in an equilibrium adjustment of the peracetic acid and acetic acid concentrations in the solution.

The analyses for these Examples also confirm that the dilute peracetic acid solutions were essentially equilibrium solutions, with respect to their peracetic acid, hydrogen peroxide and acetic acid components in the initial dilute peracetic solutions, since there was no significant shift in the respective proportions and amounts of these components in the solution over the first month of the test period. The change that occurred in the dilute peracetic acid composition of Examples 2A, 2B and 2C between their initial preparation (day 0) and one day later (day 1) suggests that the solutions were equilibrating themselves, with the equilibrium concentrations of the peracetic acid with the other solution components being reached within one day of preparation.

### Example 3

Example 3 is a laboratory-scale study in which two dilute aqueous peracetic acid solutions were prepared according to the method of this invention, containing 0.3 wt % stabilizer but with two levels of hydrogen peroxide, and separate samples were evaluated for their long term stability while being maintained at constant temperatures of 25° C. and 50° C.

The aqueous peracetic acid solutions in Examples 3A and 3B were prepared according to the general procedure described in Example 1. In addition, Examples 3A and 3B also contained sulfuric acid, introduced at a concentration of 0.24 wt % H₂SO₄. The compositions of the dilute peracetic acid solutions of Examples 3A and 3B were analyzed after their preparation for their peracetic acid, hydrogen peroxide and acetic acid concentrations, and these analyses are respectively shown in Tables 3A and 3B, in the first data row of each Table.

The stabilized dilute peracetic acid solution in Example 3A contained 0.65 wt % peracetic acid, 9.1 wt % hydrogen peroxide, 5.2 wt % acetic acid and 0.24 wt % H₂SO₄ and therefore had a mole ratio of hydrogen peroxide to acetic acid of about 3.1:1. The stabilized dilute peracetic acid solution in Example 3B, with a lesser concentration of hydrogen peroxide, contained 0.63 wt % peracetic acid, 6.7 wt % hydrogen peroxide, 7.3 wt % acetic acid and 0.24 wt % H₂SO₄ and therefore had a mole ratio of hydrogen peroxide to acetic acid of about 1.6:1. The stabilizer in both Examples was Dequest® 2010 stabilizer comprising 1-hydroxy ethylidene-1,1-diphosphonic acid, the stabilizer being used at a concentration of 0.3 wt % stabilizer.

Separate samples of the dilute peracetic acid solutions were evaluated at two constant storage temperatures, 25° C. and 50° C., for an extended period lasting over nine months. The dilute peracetic acid solution samples were periodically analyzed to determine their concentrations of peracetic acid, hydrogen peroxide and acetic acid during the storage period.

Both of the dilute peracetic acid solution samples maintained at 25° C. showed excellent stability, since the samples exhibited essentially unchanged levels of peracetic acid, hydrogen peroxide and acetic acid over a period of 295 days.

The dilute peracetic acid solution samples maintained at 50° C., a more stringent stability test temperature, demonstrated generally good stability at the two levels of hydrogen peroxide used, 0.6 and 0.9 wt % H₂O₂, with a constant stabilizer concentration of 0.3 wt % stabilizer. The periodic analyses of the dilute peracetic acid solutions maintained at a storage temperature of 50° C. are shown for Examples 3A and 3B in Tables 3A and 3B, respectively, for the 295 days storage stability test period.

TABLE 3A
Long Term Stability Evaluation at 50° C.:
Aqueous 0.65 wt % Peracetic Acid Solution with 9.1 wt % H₂O₂
Containing 0.3 wt % Dequest ® Stabilizer

| Days | Peracetic Acid | H₂O₂ | Acetic Acid |
|---|---|---|---|
| after Preparation | wt % | wt % | wt % |
| 0 | 0.65 | 9.1 | 5.2 |
| 1 | 0.60 | 9.2 | 5.2 |
| 3 | 0.61 | 9.1 | 5.2 |
| 7 | 0.61 | 9.1 | 5.2 |
| 14 | 0.60 | 9.1 | 5.2 |
| 28 | 0.60 | 9.1 | 5.2 |
| 57 | 0.62 | 9.0 | 5.2 |
| 87 | 0.59 | 8.9 | 5.3 |
| 112 | 0.57 | 8.8 | 5.3 |
| 136 | 0.60 | 8.7 | 5.3 |
| 171 | 0.58 | 8.5 | 5.3 |
| 199 | 0.54 | 8.2 | 5.4 |
| 233 | 0.57 | 7.9 | 5.4 |
| 267 | 0.49 | 7.4 | 5.4 |
| 295 | 0.46 | 6.8 | 5.4 |

TABLE 3B
Long Term Stability Evaluation at 50° C.:
Aqueous 0.63 wt % Peracetic Acid Solution with 6.7 wt % H₂O₂
Containing 0.3 wt % Dequest ® Stabilizer

| Days | Peracetic Acid | H₂O₂ | Acetic Acid |
|---|---|---|---|
| after Preparation | wt % | wt % | wt % |
| 0 | 0.63 | 6.7 | 7.3 |
| 1 | 0.60 | 6.7 | 7.3 |
| 3 | 0.61 | 6.7 | 7.3 |
| 7 | 0.59 | 6.7 | 7.3 |
| 14 | 0.58 | 6.6 | 7.4 |
| 28 | 0.57 | 6.7 | 7.3 |
| 57 | 0.56 | 6.6 | 7.4 |
| 87 | 0.56 | 6.4 | 7.4 |
| 112 | 0.54 | 6.3 | 7.4 |
| 136 | 0.57 | 4.8 | 7.6 |
| 171 | 0.58 | 6.0 | 7.5 |
| 199 | 0.55 | 5.8 | 7.6 |
| 233 | 0.46 | 5.5 | 7.6 |
| 269 | 0.40 | 5.1 | 7.7 |
| 295 | 0.36 | 4.6 | 7.7 |

The peracetic acid solution in Example 3A, which was a 0.65 wt % peracetic acid solution with 9.1 wt % H₂O₂, retained about 70% of the initial peracetic acid concentration (and about 75% of the initial hydrogen peroxide concentration) after 295 days (over 9 months) of the solution being maintained at a constant storage temperature of 50° C. In the first two months of the study in Example 3A, the initial peracetic acid solution composition, at a temperature of 50° C., remained essentially unchanged as shown by the composition data in Table 3A and thereafter still retained about 89% of the initial peracetic acid level even after 171 days at a constant 50° C. temperature.

The peracetic acid solution in Example 3B, which was a 0.63 wt % peracetic acid solution with 6.7 wt % H₂O₂, retained about 57% of the initial peracetic acid concentration (and about 69% of the initial hydrogen peroxide concentration) after 295 days (over 9 months) of the solution being maintained at a constant storage temperature of 50° C. In the first five months of the study in Example 3B, the peracetic acid solution composition in Example 3B, at a temperature of 50° C., exhibited a very slow decline in its initial composition, as shown by the composition data in Table 3B, but still retained over 90% of the initial peracetic acid level even after 171 days at a constant 50° C. temperature. However, the peracetic acid concentration began to decrease after 199 days, as shown by the lower peracetic acid concentrations measured at 233 days and thereafter.

These results for Example 3B, when compared with those of Example 3A, suggest that the higher hydrogen peroxide concentration used in Example 3A (9.1 wt % H₂O₂ vs. 6.7 wt % H₂O₂ in Example 3B) and the higher mole H₂O₂:CH₃COOH mole ratio used in Example 3A (3.1:1 vs. 1.6:1 in Example 3B) were beneficial in providing slightly better long term stability of the aqueous peracetic acid solution for Example 3A. This conclusion is based on the results of Example 3A, which performed slightly better in its peracetic acid stability towards the end of the long term stability test period, i.e., at 233, 267 and 295 days after initial preparation.

The analyses for these Examples also confirm that the dilute peracetic acid solutions were essentially equilibrium solutions, with respect to their peracetic acid, hydrogen peroxide, acetic acid and water components in the initial dilute peracetic solutions, since there was no significant shift in the respective proportions and amounts of these components in the solution over the first month of the test period. The change that occurred in the dilute peracetic acid compositions of Examples 3A and 3B between their initial preparation (day 0) and one day later (day 1) suggests that the solutions were equilibrating themselves, with the equilibrium concentrations of the peracetic acid with the other solution components being reached within one day of preparation.

### Comparative Example 1

This Comparative Example 1 evaluated the long term stability of a 0.7 wt % peracetic acid solution prepared by dilution of a stabilized concentrated (16 wt %) peracetic acid formulation. The dilute peracetic acid solution in Comparative Example 1 contained 0.73 wt % peracetic acid, 9.1 wt % hydrogen peroxide and 5.1 wt % acetic acid but only 0.02 wt % Dequest® stabilizer, the latter carried over from the initial stabilized 16 wt % peracetic acid solution that was diluted.

The dilute 0.7 wt % peracetic acid solution of this Comparative Example was prepared by dilution of a stabilized concentrated peracetic acid solution containing 16 wt % peracetic acid, 10 wt % hydrogen peroxide, 35 wt % acetic acid and 0.62 wt % Dequest stabilizer. This concentrated peracetic acid solution was the same solution as that described in Example 1, and the procedure used for its dilution was generally the same as described for Example 1, with one exception. No additional stabilizer was added during the dilution procedure. The only stabilizer present was that originally present in the concentrated peracetic acid solution, and the addition of diluent acetic acid, hydrogen peroxide and water reduced the stabilizer concentration from 0.62 wt % to 0.02 wt % stabilizer.

The dilute peracetic acid solution in Comparative Example 1 contained 0.73 wt % peracetic acid, 9.1 wt % hydrogen peroxide, 5.1 wt % acetic acid and 0.02 wt % residual Dequest stabilizer. The peracetic acid solution therefore had a mole ratio of hydrogen peroxide to acetic acid of about 3.1:1.

Separate samples of the dilute peracetic acid solution were evaluated at two constant storage temperatures, 25° C. and 50° C., for an extended period lasting over four months. The dilute peracetic acid solution samples were periodically analyzed to determine their concentrations of peracetic acid, hydrogen peroxide and acetic acid during the storage period.

The dilute peracetic acid solution samples maintained at 25° C. showed excellent stability, with the composition analyses exhibiting essentially unchanged levels of peracetic acid, hydrogen peroxide and acetic acid over a period of 136 days.

The periodic analyses of the dilute peracetic acid solution maintained at a storage temperature of 50° C. are shown for Comparative Example 1 in Table C-1 for the 136 days storage stability test period.

TABLE C-1
Long Term Stability Evaluation at 50° C.:
Aqueous 0.7 wt % Peracetic Acid Solution
Containing 0.02 wt % Residual Stabilizer

| Days | Peracetic Acid | H₂O₂ | Acetic Acid |
|---|---|---|---|
| after Preparation | wt % | wt % | wt % |
| 0 | 0.73 | 9.1 | 5.1 |
| 1 | 0.71 | 9.2 | 5.2 |
| 3 | 0.70 | 9.2 | 5.2 |
| 7 | 0.68 | 9.1 | 5.2 |
| 14 | 0.68 | 9.1 | 5.2 |
| 28 | 0.69 | 9.1 | 5.2 |
| 57 | 0.66 | 8.8 | 5.2 |
| 87 | 0.38 | 7.1 | 5.4 |
| 112 | 0.22 | 3.7 | 5.3 |
| 136 | 0.15 | 1.7 | 5.2 |

At the constant storage temperature of 50° C., the peracetic acid solution in Comparative Example 1, a 0.7 wt % peracetic acid solution with 0.02 wt % residual Dequest® stabilizer, exhibited good stability for the first two months of the solution being maintained at 50° C., retaining about 90% of the initial peracetic acid concentration (and about 78% of the initial hydrogen peroxide concentration) after 57 days.

After two months, however, stability of the peracetic acid solution deteriorated rapidly, as shown by the peracetic acid concentration data in Table C-1 for the solution at 87, 112 and 136 days at a constant storage temperature of 50° C. At the end of the 136 day storage period, the peracetic acid solution contained only 20% of its initial peracetic acid concentration (and less than 20% of the initial hydrogen peroxide concentration), demonstrating poor long term stability for this peracid acid solution. These results suggest that the 0.02 wt % level of stabilizer present in this peracetic acid solution was inadequate to provide good long term stability.

### Comparative Example 2

This Comparative Example 2 evaluated the long term stability of a dilute peracetic acid formulation described in the prior art as having “excellent shelf life.” This peracetic acid formulation is one disclosed in Greenspan et al., in Proc. 42nd Ann. Mtg. Chem. Spec. Man. Assn. December 1955, pp. 59-64 at p. 61, and is described as containing 1.0 wt % peracetic acid, 14.5 wt % acetic acid, 5.0 wt % hydrogen peroxide, 78.5 wt % water and 1.0 wt % sulfuric acid. The mole ratio of hydrogen peroxide to acetic acid for this peracetic acid solution was therefore about 0.6:1. No stabilizer was present in the preferred peracetic acid formulation disclosed by Greenspan et al.

Greenspan et al. measured the stability of this dilute peracetic acid formulation at a temperature of 86° F. (30° C.) and found that only 2.7% of its initial peracetic acid content was lost over 81 days (see Table II, p. 61), in contrast to a “standard 1 percent peracetic acid” solution, with pH 2.5, that lost 50% of its initial peracetic acid content after 6 days.

For this Comparative Example 2, a nominal 1 wt % peracetic acid solution was prepared in the laboratory at a temperature of 25° C. using the relative weight amounts of components described above just as for the Greenspan et al. controlled formulation 1% peracetic acid. This 1 wt % peracetic acid solution was observed to be not equilibrated. Its composition shifted immediately after its preparation to yield a solution containing 2.0 wt % peracetic acid, 14.2 wt % acetic acid, and 5.2 wt % hydrogen peroxide, for the solution prepared at 25° C. When the temperature of the solution was increased to 50° C., its composition shifted further to yield a solution containing 1.5 wt % peracetic acid, 14.2 wt % acetic acid, and 5.0 wt % hydrogen peroxide.

As in the other Examples, separate samples of the dilute peracetic acid solutions for this Comparative Example 2 were evaluated at two constant storage temperatures, 25° C. and 50° C., for a period lasting over three months. The dilute peracetic acid solution samples were periodically analyzed to determine their concentrations of peracetic acid, hydrogen peroxide and acetic acid during the storage period. The periodic analyses of the dilute peracetic acid solutions maintained at the storage temperatures of 25° C. and 50° C. are shown for Comparative Example 2 in Tables C-2A and C-2B, respectively, for the 113 day storage stability test period.

TABLE C-2A
Long Term Stability Evaluation at 25° C.:
Greenspan et al. Nominal 1 wt % Peracetic Acid Solution

| Days | Peracetic Acid | H₂O₂ | Acetic Acid |
|---|---|---|---|
| after Preparation | wt % | wt % | wt % |
| 0 | 2.0 | 5.2 | 14.2 |
| 2 | 1.4 | 5.0 | 14.1 |
| 8 | 0.3 | 5.0 | 15.1 |
| 12 | 0.4 | 5.0 | 15.0 |
| 44 | 1.2 | 5.0 | 14.5 |
| 80 | 0.9 | 4.9 | 14.6 |
| 113 | 0.2 | 4.9 | 15.2 |

TABLE C-2B
Long Term Stability Evaluation at 50° C.:
Greenspan et al. Nominal 1 wt % Peracetic Acid Solution

| Days | Peracetic Acid | H₂O₂ | Acetic Acid |
|---|---|---|---|
| after Preparation | wt % | wt % | wt % |
| 0 | 1.5 | 5.2 | 14.1 |
| 2 | 1.4 | 5.0 | 14.1 |
| 7 | 1.7 | 5.0 | 14.0 |
| 12 | 0.6 | 4.9 | 14.8 |
| 44 | 0.4 | 5.0 | 15.0 |
| 80 | 0.6 | 3.0 | 14.7 |
| 113 | no analysis possible | 2.1 | no analysis possible |

As shown by the data in Table C-2B above, the stability of the Greenspan et al. nominal 1 wt % peracetic acid solution at the more stringent storage temperature of 50° C. was poor, with the peracetic acid concentration dropping to 40% of its initial peracetic acid concentration after only 12 days.

The results for the sample stored at 25° C., shown in Table C-2A above, were also unsatisfactory, with the peracetic acid concentration being highly variable over the 113 day storage test period. The peracetic acid concentration stored at 25° C. dropped to 20% of its initial peracetic acid concentration after only 12 days but recovered somewhat after 44 days of storage, before continuing to decline significantly again.

It will be appreciated by those skilled in the art that changes could be made to the embodiments described above without departing from the broad inventive concept thereof. It is understood, therefore, that this invention is not limited to the particular embodiments disclosed but is intended to cover modifications within the spirit and scope of the present invention as defined by the appended claims.

What is claimed is:

1. A method of stabilizing a dilute peracid solution comprising
adjusting the concentration of hydrogen peroxide in an aqueous organic peracid solution, containing a peroxycarboxylic acid, hydrogen peroxide, and corresponding carboxylic acid and, further, containing at least about 0.05 to less than about 5 wt % of the organic peroxycarboxylic acid, to provide
a hydrogen peroxide concentration of less than about 20 wt % H₂O₂ and
a mole ratio of hydrogen peroxide to carboxylic acid in excess of at least about 1.5:1 hydrogen peroxide:carboxylic acid; and
introducing a stabilizer into the aqueous peracid solution, in an amount of about 0.05 wt % to about 5 wt % stabilizer based on the weight of the peracid solution.

2. The method of claim 1 wherein the peroxycarboxylic acid is a C₁ to C₁₂ peroxycarboxylic acid selected from the group consisting of monocarboxylic peracids and dicarboxylic peracids.

3. The method of claim 1 wherein the peroxycarboxylic acid is a C₂ to C₅ peroxycarboxylic acid selected from the group consisting of monocarboxylic peracids and dicarboxylic peracids.

4. The method of claim 1 wherein the aqueous peracid solution is equilibrated with respect to its peracid, hydrogen peroxide, carboxylic acid and water components.

5. The method of claim 1 wherein the peracid concentration is at least about 0.1 wt % peracid.

6. The method of claim 1 wherein the concentration of hydrogen peroxide in the aqueous peracid solution is adjusted to provide less than about 10 wt % H₂O₂.

7. The method of claim 1 wherein the hydrogen peroxide concentration in the aqueous peracid solution is at least about 2 wt % H₂O₂.

8. The method of claim 1 wherein the stabilizer is selected from the group consisting of
organic phosphonic acids; amine-substituted phosphonic acids; alkyleneaminomethylene phosphonic acids; carboxylic acid substituted N-containing heterocyclics; aminopolycarboxylic acids; polyaminocarboxylic acids; tin-based compounds; phosphoric acids; alkylbenzene sulfonates with 6-18 carbon atoms; alkyl sulfates; and water-soluble salts of these acids.

9. The method of claim 1 wherein the stabilizer comprises 1-hydroxy ethylidene-1,1-diphosphonic acid.

10. A method of stabilizing a dilute peracetic acid solution comprising
adjusting the concentration of hydrogen peroxide in an aqueous peracetic acid solution, containing hydrogen peroxide, acetic acid and at least about 0.05 to less than about 5 wt % peracetic acid, to provide
a hydrogen peroxide concentration of less than about 20 wt % H₂O₂ and
a mole ratio of hydrogen peroxide to acetic acid of at least about 1.5:1 H₂O₂:CH₃COOH; and
introducing a stabilizer into the aqueous peracetic acid solution, in an amount of about 0.05 wt % to about 5 wt % stabilizer based on the weight of the peracid solution.

11. The method of claim 10 wherein the aqueous peracetic acid solution is equilibrated with respect to its peracetic acid, hydrogen peroxide, acetic acid and water components.

12. The method of claim 10 wherein the peracetic acid concentration is less than about 3 wt % peracetic acid.

13. The method of claim 10 wherein the peracetic acid concentration is at least about 0.1 wt % peracetic acid.

14. The method of claim 10 wherein the concentration of hydrogen peroxide in the aqueous peracetic acid solution is adjusted to provide less than about 15 wt % H₂O₂.

15. The method of claim 10 wherein the concentration of hydrogen peroxide in the aqueous peracetic acid solution is adjusted to provide less than about 10 wt % H₂O₂.

16. The method of claim 10 wherein the hydrogen peroxide concentration in the aqueous peracetic acid solution is at least about 2 wt % H₂O₂.

17. The method of claim 10 wherein the concentration of hydrogen peroxide in the aqueous peracetic acid solution is adjusted to provide a mole ratio of hydrogen peroxide to acetic acid of at least about 2:1 H₂O₂:CH₃COOH.

18. The method of claim 10 wherein the stabilizer is selected from the group consisting of organic phosphonic acids; amine-substituted phosphonic acids; alkyleneaminomethylene phosphonic acids; carboxylic acid substituted N-containing heterocyclics; aminopolycarboxylic acids; polyaminocarboxylic acids; tin-based compounds; phosphoric acids; alkylbenzene sulfonates with 6-18 carbon atoms; alkyl sulfates; and water-soluble salts of these acids.

19. The method of claim 10 wherein the stabilizer comprises 1-hydroxy ethylidene-1,1-diphosphonic acid.

20. The method of claim 10 wherein the amount of stabilizer introduced is about 0.1 wt % to about 3 wt % stabilizer, based on the weight of the aqueous peracetic acid solution.

21. The method of claim 10 which further comprises utilizing a purified water source for preparation of the peracetic acid solution, the water source selected from the group consisting of deionized water and distilled water.

22. The method of claim 10 which further comprises utilizing purified components for the preparation of the peracetic acid solution.

23. A method of stabilizing a dilute peracetic acid solution comprising
adjusting the concentration of hydrogen peroxide in an aqueous peracetic acid solution, containing hydrogen peroxide, acetic acid and about 0.2 to about 3 wt % peracetic acid, to provide
a hydrogen peroxide concentration of about 2 wt % to less than about 10 wt % H₂O₂ and
a mole ratio of hydrogen peroxide to acetic acid of at least about 1.5:1 H₂O₂:CH₃COOH; and
introducing a stabilizer comprising 1-hydroxy ethylidene-1,1-diphosphonic acid into the aqueous peracetic acid solution, the stabilizer being introduced in an amount of about 0.1 to about 3 wt % based on the weight of the solution.

24. The method of claim 23 wherein the concentration of hydrogen peroxide in the aqueous peracetic acid solution is adjusted to provide a mole ratio of hydrogen peroxide to acetic acid of at least about 2:1 H₂O₂:CH₃COOH.

25. The method of claim 23 which further comprises utilizing a purified water source for preparation of the peracetic acid solution, the water source selected from the group consisting of deionized water and distilled water.

26. A stabilized dilute aqueous peracetic acid solution comprising peracetic acid, hydrogen peroxide and acetic acid and containing about 0.5 to about 3 wt % peracetic acid, about 2 wt % to less than about 10 wt % hydrogen peroxide, and a stabilizer in an amount of about 0.1 to about 3 wt % based on the weight of the solution, wherein the mole ratio of hydrogen peroxide to acetic acid is at least about 1.5:1 H₂O₂:CH₃COOH.

27. The stabilized dilute aqueous peracetic acid solution of claim 26 wherein the aqueous peracetic acid solution is equilibrated with respect to its peracetic acid, hydrogen peroxide, acetic acid and water components.

28. The stabilized dilute aqueous peracetic acid solution of claim 26 wherein the stabilizer comprises 1-hydroxy ethylidene-1,1-diphosphonic acid.
